(19) United States
(12) Patent Application Publication
(10) Pub. No.: US 2007/0235041 A1
(43) Pub. Date: Oct. 11, 2007

(54) "Stampede" protection belt

(76) Inventor: Magnar J. Grande, Metairie, LA (US)

Correspondence Address: Magnar J. Grande, Apt. 209, 3728 West Esplanade Ave. South, Metairie, LA, 70002, US

(21) Appl. No.: 11/390,328

(22) Filed: Mar. 28, 2006

(51) Int. Cl. A61F 5/37

(52) U.S. Cl. 128/870

(57) ABSTRACT

The invention provides a method of saving the lives of people by providing a protective “belt” around the chest area so that a person can breath freely in a stampede situation.

### BACKGROUND OF THE INVENTION

The invention relates to means and methods of saving lives in a situation where large gatherings of people are present, such as at concerts, religious gatherings or seasonal celebrations such as Mardi Gras in New Orleans and Brazil.

It is well known that thousands of people have been killed in the crush of a stampede due to the fact that one can breathe air out of the lungs but find it is difficult or impossible to expand the chest in order to take in air due to the compound physical pressure of the people, consequently losing consciousness, resulting in being seriously injured or trampled to death.

### DESCRIPTION OF DRAWING

The above object, together with its described features of the Invention, will be apparent in light of the details of construction and use of the Belt.

FIG. 1. in the drawing depicts a front elevation view of the Belt.

FIG. 2. in the drawing represents a cross section view, taken along the line “A-A” of FIG. 1, that also shows a shoulder strap arrangement.

FIG. 3. in the drawing represents a side elevation view of the Belt.

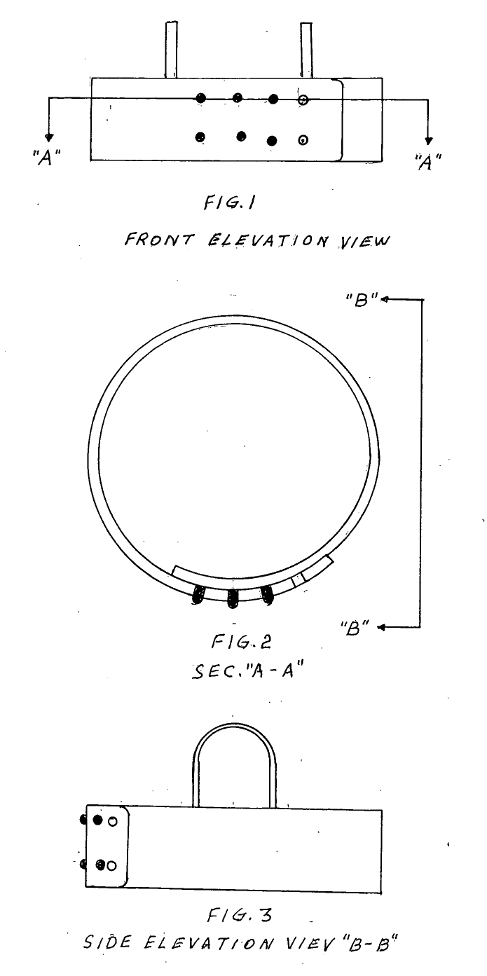

Referring to FIGS. 1, 2 and 3 of the drawing, the Belt can be made by molding of an especially hardened plastic material or a softer plastic material with a steel inlay.

What I claim in my Invention is:

1. means of allowing individual humans to breath freely under conditions where people gather in large numbers and assert intolerable physical pressure on each other in a stampede situation.
